SEQUENCE LISTING (12 sequences)

SEQ ID NO: 1 - RNA, 10 nt, Artificial Sequence
  binding site
rccaccaugg

SEQ ID NO: 2 - protein, 20 aa, Artificial Sequence
  peptide binding to green fluorescent protein
CSGFRAMWLYRNWESQVEAT

SEQ ID NO: 3 - protein, 20 aa, Artificial Sequence
  peptide binding to green fluorescent protein
CSGWNHVIYEGTRYNWFRDS

SEQ ID NO: 4 - protein, 20 aa, Artificial Sequence
  peptide binding to green fluorescent protein
CSGPYGTHFMYKSGGWRAIY

SEQ ID NO: 5 - protein, 18 aa, Artificial Sequence
  peptide binding to thioredoxin
LVTDETISYFRDQDAEIG

SEQ ID NO: 6 - protein, 20 aa, Artificial Sequence
  peptide binding to thioredoxin
IIHWKQYHADMLLLEWKGSC

SEQ ID NO: 7 - protein, 20 aa, Artificial Sequence
  peptide binding to thioredoxin
TPPLSSRWEHWFNMQNKGSC

SEQ ID NO: 8 - protein, 20 aa, Artificial Sequence
  peptide binding to thioredoxin
WWYTLGEQIPRWPQKGWGSC

SEQ ID NO: 9 - protein, 20 aa, Artificial Sequence
  peptide binding to thioredoxin
IQEWSNMVIWQETYRKIGSC

SEQ ID NO: 10 - protein, 20 aa, Artificial Sequence
  peptide binding to thioredoxin
PGKDRADWKHYGNYYPTGSC

SEQ ID NO: 11 - protein, 18 aa, Artificial Sequence
  linker peptide
TQPPSHGSAGSAAGSGEF

SEQ ID NO: 12 - protein, 12 aa, Artificial Sequence
  linker peptide
GSAGSAAGSGEF

(19) United States
(12) Patent Application Publication
(10) Pub. No.: US 2012/0316078 A1
(43) Pub. Date: Dec. 13, 2012

(54) METHODS AND MATERIALS FOR PRODUCING POLYPEPTIDES IN VITRO

(76) Inventors: Kathryn F. Sykes, Tempe, AZ (US); Zhan-Gong Zhao, Tucson, AZ (US); Stephen Albert Johnston, Tempe, AZ (US); Andrey Loskutov, Gilbert, AZ (US)

(21) Appl. No.: 13/492,304

(22) Filed: Jun. 8, 2012

Related U.S. Application Data
(60) Provisional application No. 61/494,527, filed on Jun. 8, 2011.

(51) Int. Cl. C12P 21/00; G01N 21/64; C40B 30/04; C40B 50/06

(52) U.S. Cl. 506/9; 435/69.7; 506/26; 435/7.8; 435/69.6

(57) ABSTRACT

Methods for producing polypeptides in vitro are described that use free template nucleic acids that are not immobilized on a substrate. Polypeptides that are produced can be captured on particles without the use of capture agents and can be used to produce polypeptide arrays.

### CROSS-REFERENCE TO RELATED APPLICATIONS

This application claims the benefit of U.S. Provisional Application Ser. No. 61/494,527, filed Jun. 8, 2011. The disclosure of the prior application is considered part of (and is incorporated by reference in) the disclosure of this application.

### TECHNICAL FIELD

This invention relates to methods and materials for producing polypeptides in vitro, and more particularly to using free template nucleic acids that are not immobilized on a substrate to produce polypeptides, which are captured on particles during their synthesis.

### BACKGROUND

Two platforms for producing protein arrays are micro spotting and in situ self-assembly. Micro spotting allows high volume production but is burdened by the tedious process of protein expression and purification, complicated by the wide variation in protein solubilities, and further, complicated by the tendency of proteins to unfold when immobilized onto a solid surface due to hydrophobic interaction between internal hydrophobic residues and the solid surface. The in situ self-assembling platform relies exclusively on an affinity tag fused to each of the target proteins for immobilization. The fusion proteins are synthesized in situ on a cDNA-patterned array surface, and are captured by a fusion-tag specific antibody spotted on the same spot as the immobilized target gene. A major disadvantage associated with this platform is that the yield and quality of expression cannot be easily evaluated on the fixed spots and, therefore, the quality of the array cannot be assured. Furthermore, these proteins cannot be used in any other assay, individually or in subsets, since they are fixed in toto to the slide.

### SUMMARY

This document is based on the discovery of an efficient process for producing and purifying polypeptides. The methods described herein are particularly useful for uniformly producing, purifying, and presenting functionally soluble polypeptides in a suspension for use in a number of formats such as an array, in an integrated process. Free template nucleic acids encoding a polypeptide containing a tag (e.g., a fluorescent tag, chaperone tag, peptide tag, or charged amino acid tag) are used, along with transcription and translation effectors, to produce polypeptides that can be captured on a particle as the nascent chains emerge from the ribosome. In some embodiments, the polypeptide is captured on the surface of a particle without the need for an agent. In some embodiments, the particle contains an agent (e.g., antibody, aptamer, or synbody) that has binding affinity for the tag on the polypeptide. Particles containing the captured polypeptide can be directly spotted onto a solid surface (e.g., a glass slide) or used individually or in pools in other suspension assays without further purification. For example, the particles containing the captured polypeptides can be used in any assay requiring fluidity, such as enzyme assays, microtiter plate screens, micro-array probings, or immunizations of animals.

In one aspect, this document features a method for producing a polypeptide in vitro. The method includes producing the polypeptide using a free template nucleic acid, a transcription effector, and a translation effector in the presence of a particle (e.g., a magnetic particle or a hydrophobic particle); wherein the free template nucleic acid encodes the polypeptide and is capable of being transcribed and translated; and wherein the polypeptide includes a tag (e.g., a fluorescent tag such as a fluorescent tag at the C-terminus of the polypeptide or thioredoxin); and capturing the polypeptide on the particle (e.g., via hydrophobic interaction between the polypeptide chain and the surface of the particle) during synthesis.

In another aspect, this document features a method for producing a polypeptide in vitro. The method includes producing the polypeptide using a free template nucleic acid, a transcription effector, and a translation effector in the presence of a particle (e.g., a magnetic particle); the free template nucleic acid encoding the polypeptide and capable of being transcribed and translated; wherein the polypeptide includes a tag (e.g., a fluorescent tag such as a fluorescent tag at the C-terminus of the polypeptide). The particle can include a peptide or synbody having binding affinity for the tag (e.g., fluorescent tag); and capturing the polypeptide on the particle during synthesis via binding of the tag on the polypeptide to the peptide (e.g., a peptide 10 to 30 amino acids in length) or synbody on the particle.

In the methods described herein, the polypeptide can be a membrane protein. The polypeptide can be a hydrophobic polypeptide. The fluorescent tag can be green fluorescent protein (GFP) or enhanced GFP, blue fluorescent protein, cyan fluorescent protein, red fluorescent protein, or yellow fluorescent protein.

The methods described herein further can include separating the particle including the bound polypeptide from the transcription and translation effectors. The transcription effector can be a prokaryotic RNA polymerase such as a T7, T3, or SP6 RNA polymerase. The translation effector can be a prokaryotic or eukaryotic cell lysate or extract. For example, the prokaryotic cell lysate or extract can be an Escherichia coli extract. The eukaryotic cell lysate or extract can be a human cell lysate or extract, rabbit reticulocyte lysate, or wheat germ extract.

The methods described herein further can include detecting fluorescence of the polypeptide bound to the particles or measuring the amount of fluorescence to quantitate the amount of polypeptide produced using the transcription and translation effectors. The amount of fluorescence can be measured using a microfluidic device, or a microarray reader or microscope capable of detecting fluorescence.

The methods described herein further can include spotting the particles comprising the bound polypeptide onto an amine reactive array surface or microchip.

In some embodiments, a plurality of different template nucleic acids is provided; wherein each different template nucleic acid encodes a polypeptide having a different fluorescent tag. In some embodiments, a plurality of different template nucleic acids and a plurality of different particles are provided, wherein each different template nucleic acid encodes a polypeptide having a different fluorescent tag; and wherein each different particle has binding affinity for one fluorescent tag. In some embodiments, a plurality of different template nucleic acids is provided; wherein each different template nucleic acid encodes a polypeptide having a different fluorescent tag; and wherein each particle has binding affinity for two or more fluorescent tags.

In another aspect, this document features a method for producing a polypeptide in vitro that includes producing the polypeptide using a free template nucleic acid, a transcription effector, and a translation effector in the presence of a particle; wherein the free template nucleic acid encodes the polypeptide and is capable of being transcribed and translated; wherein the polypeptide includes a fluorescent tag; capturing the polypeptide on the particle; and measuring the amount of fluorescence to quantitate the amount of polypeptide produced using the transcription and translation effectors. The amount of fluorescence can be measured using a microarray reader or microscope capable of detecting fluorescence. The amount of fluorescence can be detected by a microfluidic device.

This document also features a method for producing a polypeptide in vitro. The method includes producing the polypeptide using a free template nucleic acid, a transcription effector, and a translation effector in the presence of a particle; the free template nucleic acid encoding the polypeptide and capable of being transcribed and translated; wherein the polypeptide includes a fluorescent tag and the particle includes a peptide or synbody having binding affinity for the fluorescent tag; capturing the polypeptide on the particle via binding of the tag on the polypeptide to the peptide or synbody on the particle; and measuring the amount of fluorescence to quantitate the amount of polypeptide produced using the transcription and translation effectors. The amount of fluorescence can be measured using a microarray reader or microscope capable of detecting fluorescence. The amount of fluorescence can be detected by a microfluidic device.

In another aspect, this document features a method for producing a polypeptide in vitro. The method includes producing the polypeptide using a free template nucleic acid, a transcription effector, and a translation effector in the presence of a particle; the free template nucleic acid encoding the polypeptide and capable of being transcribed and translated; wherein the polypeptide comprises a tag (e.g., thioredoxin) and the particle includes a peptide or synbody having binding affinity for the tag; and capturing the polypeptide on the particle via binding of the tag on the polypeptide to the peptide or synbody on the particle.

In yet another aspect, this document features a method for producing a polypeptide in vitro that includes producing the polypeptide using a free template nucleic acid, a transcription effector, and a translation effector in the presence of a particle; the free template nucleic acid encodes the polypeptide and is capable of being transcribed and translated; and capturing the polypeptide on the particle. The polypeptide can include a tag.

This document also features a method for producing a polypeptide in vitro. The method includes producing the polypeptide using a free template nucleic acid, a transcription effector, and a translation effector in the presence of a particle; the free template nucleic acid encoding the polypeptide and capable of being transcribed and translated; wherein the polypeptide includes a tag and the particle includes an agent having binding affinity for the tag; and capturing the polypeptide on the particle via binding of the tag on the polypeptide to the agent on the particle. The agent can be an antibody or antigen-binding fragment thereof (e.g., Fab, F(ab′)₂, Fv, or single chain Fv (scFv) fragment). The tag can be thioredoxin or a fluorescent protein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic of an in vitro transcription and translation reaction using free template nucleic acid and particles without capture agent. As polypeptides are newly synthesized, the extended chains attach directly to the hydrophobic surface of the magnetic beads. All other lysate components remain unbound and are washed away.

FIG. 2 is a schematic diagram of the immobilization process using a magnet based slide holder.

FIG. 3 is a representation of a sodium dodecyl sulfate (SDS)-polyacrylamide gel electrophoresis (PAGE) Coomassie stained gel. Lanes 1 and 2 show concentration standards (BSA). Lanes 3-5 are the washed beads following in vitro transcription and translation (IVTT) reactions. Lanes 6-8 are the supernatant of the IVTT reaction before washing. These wells display the unbound proteins of the reaction mix. The numbers 1-8 in the gel refer to the following IVTT templates: 1, no template; 2—FTT0472A (33 kDa); 3 and 6—ASFV127 (41 kDa); 4 and 7—ASFV142-1 (38 kDa); 5 and 8—FTT1656A (44 kDa). The blue dots mark the position corresponding to the calculated molecular weight of the fusion protein. There is no polypeptide band corresponding to the target protein molecular weight in the supernatant lanes, indicating quantitative capture by the beads.

FIG. 4 is a representation of SDS-PAGE Coomassie stained gels (upper panels) and scans of the same gels on a Phosphorimager (Typhoon™) to measure radioisotope emissions (lower panels) of 20 FTT predicted membrane proteins synthesized using the New (N) or Standard (S) methods of synthesizing and purifying proteins in vitro. Each N lane was loaded with 10% of the IVTT reaction, whereas each S lane was loaded with 20% of the reaction to facilitate visualization of the lower yielding reactions. Lanes 1: FTT1724A, MPID-027 (48.4 kDa); 2: FTT1724B, MPID-027 (40.1 kDa); 3: FTT0583B, MPID-028 (47.1 kDa); 4: FTT1156A, MPID-024 (49.4 kDa); 5: FTT1156B, MPID-024 (50.2 kDa); 6:FTT1258A, MPID-025 (47.8 kDa); 7: FTT1573A, MPID-026 (48.5 kDa); 8:FTT1573B, MPID-026 (50.2 kDa); 9:FTT1573C, MPID-026 (47.1 kDa); 10: FTT0831A (43.5 kDa); 11:FTT0831B (42.7 kDa); 12:FTT1525A, MPID-034 (48.5 kDa); 13: FTT0918A, MPID-029 (47.6 kDa); 14: FTT0918B, MPID-029 (48.4 kDa); 15: FTT0919A, MPID-030 (44.6 kDa); 16: FTT0919B, MPID-030 (44.6 kDa); 17: FTT1459A (50.5 kDa); 18: FTT1416A, MPID-033 (29.2 kDa); 19: FTT0805A, MPID-036 (40.5 kDa); 20: FTT0805B, MPID-036 (41.1 kDa).

FIG. 5 is a scanned image of IVTT polypeptides that were captured on particles then spotted onto aminosilane-coated glass slides. GFP fluorescence was detected using a Typhoon™ imaging system. Typhoon™ imaging was performed in the fluorescence mode with PMT voltage—500V at medium sensitivity, emission 526 SP (short-pass) nm filter/Blue (488 nm).

FIG. 6 is a scanned image of IVTT reactions that were spotted on an aminosilane-coated glass slide. Fluorescence levels were determined using a Typhoon™ imaging system while the spot was still wet (left panel), after it had been allowed to dry (middle panel), and after it had been rewet by addition of 2 μl of 1× phosphate buffered saline (right panel). The fluorescence levels were the same for all samples. Typhoon™ imaging was performed in the fluorescence mode with PMT voltage—500V at high sensitivity, emission 526 SP nm filter/Blue (488 nm).

FIG. 7 is a representation of an aminosilane functionalized slide acoustically printed with 1 mm magnetic beads bound to in vitro synthesized green fluorescence protein (GFP). Printing efficiency was evaluated on a Perkin Elmer scanner at 470 nm excitation and 509 nm emission wavelengths. GFP integrity was maintained through production, purification, and printing.

### DETAILED DESCRIPTION

In general, this disclosure features methods for producing polypeptides in vitro using free template nucleic acids to produce polypeptides that can be captured on particles during synthesis. In some embodiments, polypeptides are captured in their native form. Any polypeptides can be produced, soluble or membrane, hydrophilic, amphiphilic or hydrophilic, or otherwise, using the methods described herein.

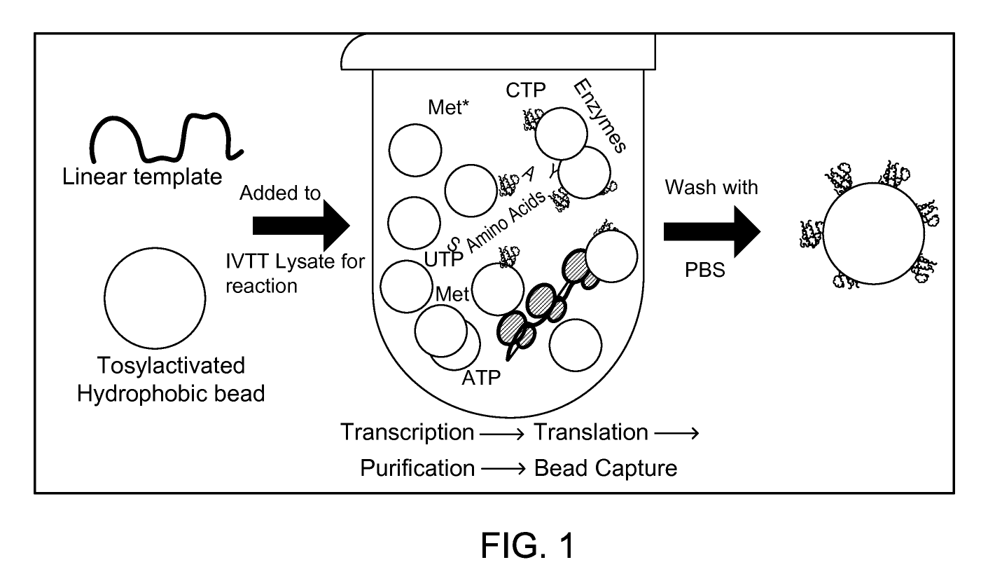

In some embodiments, the polypeptides are captured on particles without the use of capture agents. Commercially available hydrophobic, magnetic micro-bead surfaces were adapted for the immobilization of target polypeptides during their ribosomal synthesis. As shown in FIG. 1, these beads can be added to the in vitro transcription/translation (IVTT) reaction; the nascent polypeptide chains bind to the bead surfaces with exceptional selectively, using no other capture agent. The polypeptide chains remain attached to the beads such that they can be easily pipetted and used in any suspension assay, and even directly printed onto microarray slides. In addition to avoiding the expense of monoclonal capture antibodies, the samples are not contaminated with immunoglobulin or peptide tag ligands.

Using the methods described herein, high-density arrays can be rapidly and inexpensively produced in high volume. Such arrays can be used, for example, for proteomic studies and in high throughput biomedical screening technologies for drug, diagnostic, or vaccine discovery. In one embodiment, the methods described herein can be used to produce microarrays displaying natively folded pathogen proteins that can be used, for example, in immunoreactive-antigen profiling with sera from infected humans or animals. Immunogens then can be evaluated as vaccine candidates in protection assays. Since protective or therapeutic antibodies are often neutralizing, and frequently recognize conformational epitopes, the ability to query sera on folded proteins can facilitate analyses of neutralizing antibodies.

Unlike current protein arrays, the protein synthesis, purification, and printing approaches described herein can be designed to i) maximize proteome representation, ii) maximize the integrity of each protein such that both linear and non-linear, and conformational determinants can be queried, and/or iii) read out the folded state of each protein as it is positioned on the array. Furthermore, the methods described herein allow for consistency of protein behavior and attachment at each location within the array, maximizing the quantitative power of the analyses.

### Free Template Nucleic Acids

The methods for producing polypeptides described herein use free template nucleic acids. “Free template nucleic acid” refers to a nucleic acid that is not immobilized on, or bound to, a solid substrate such as a particle. The term “nucleic acid” refers to both RNA and DNA, including cDNA, genomic DNA, synthetic DNA, and DNA (or RNA) containing nucleic acid analogs. Nucleic acids can have any three-dimensional structure. A nucleic acid can be circular or linear, and double-stranded or single-stranded.

Suitable template nucleic acids encode one or more polypeptides. “Polypeptide” and “protein” are used interchangeably herein and mean any peptide-linked chain of amino acids, regardless of length or post-translational modification. A free template nucleic acid can encode any polypeptide, including, for example, hydrophobic polypeptides, membrane proteins and antibodies. In one embodiment, the template nucleic acid contains a plurality of open reading frames, e.g., the sequence is dicistronic or polycistronic. Thus, in some embodiments, a template nucleic acid can have a single open reading frame such that one particular polypeptide is produced. In some embodiments, a template nucleic acid can have two open reading frames such that two particular polypeptides are produced. In some embodiments, a template nucleic acid can have three or more open reading frames such that three particular polypeptides are produced. In some embodiments, the template nucleic acid contains two open reading frames linked together such that a fusion protein is produced.

In some embodiments, for each open reading frame, the template nucleic acid also encodes a tag such that the tag is fused to the N or C-terminus of the encoded polypeptide. For example, the template nucleic acid can encode a polypeptide having a tag at its C-terminus. In some embodiments, a plurality of different template nucleic acids is provided, where each different template nucleic acid encodes a polypeptide having a different tag.

In some embodiments, the tag is thioredoxin. The sequence of thioredoxin has been determined for many species, including, for example, mouse, human, rat, and horse. See for example, GenBank Accession Nos. NM_{—}011660, NM_{—}003329, X14878, and NM_{—}001081813, respectively. In some embodiments, the tag is a fluorescent tag such as red fluorescent protein or green fluorescent protein (GFP). For example, the tag can be a red fluorescent protein such as mCherry, tdTomato, mStrawberry, or J-Red (where m refers to monomer and td refers to tandem dimer). See, Shaner et al., Nat. Biotechnol., 22(12):1567-72 (2004). In some embodiments, the tag is GFP or a variant of GFP that has a modified excitation and fluorescence profile. The nucleotide and amino acid sequence of GFP from Aequorea victoria is set forth in GenBank under Accession No. CQ878914.1 and CAA58789, respectively. See U.S. Pat. Nos. 5,491,084 and 6,146,826, and WO 95/07463. For example, enhanced GFP, a blue fluorescent protein (FP), a cyan FP, or a yellow FP can be used as a tag. Such variants have one or more mutations relative to GFP. For example, enhanced GFP contains F64L and 565T mutations. Emerald FP contains F64L, 565T, S72A, N149K, M153T, and 1167T mutations. Yellow-green FP variants that can be used as tags include EYFP (565G, V68L, S72A, and T203Y mutations), mYFP (565G, V68L, Q69K, S72A, T203Y, and A206K mutations), citrine (565G, V68L, Q69M, S72A, and T203Y mutations), mCitrine (565G, V68L, Q69M, S72A, T203Y, and A206K mutations), Venus (F46L, F64L, 565G, V68L, S72A, M153T, V163A, 5175G, and T203Y mutations), and YPet (F46L, 147L, F64L, 565G, S72A, M153T, V163A, 5175G, T203Y, 5208F, V224L, H231E, and D234N mutations). Cyan FP variants that can be used as tags include ECFP (F64L, 565T, Y66W, N1491, M153T, and V163A mutations), mCFP (F64L, 565T, Y66W, N1491, M153T, V163A, and A206K mutations), Cerulean (F64L, 565T, Y66W, S72A, Y145A, H148D, N1491, M153T, and V163A mutations), and CyPet (T9G, V11I, D19E, F64L, 565T, Y66W, A87V, N1491, M153T, V163A, 1167A, E172T, and L194I mutations). See, Shaner et al., Nat. Methods, 2(12): 905-909 (2005). Pédelacq et al. (Nat. Biotechnol., 24(1):79-88 (2006)) describe superfolder GFP, a mutant GFP that folds with high efficiency, even if the fused polypeptide does not. Waldo et al. (Nat. Biotechnol. 17(7):691-5 (1999)) describe another mutant, the reporter GFP, that can be used for the purpose of determining whether the fused target polypeptide is folded or not. GFP and other fluorescent proteins do not require additional proteins, substrates, or cofactors in order to fluoresce. Fluorescent proteins are particularly useful tags as the amount of polypeptide produced using the methods described herein can be normalized based on the amount of fluorescence. In addition, fluorescent proteins can be used in determining the integrity and folded (native) state of the polypeptides produced as only native fluorescent proteins will fluoresce.

Template nucleic acids also include suitable translation, or transcription and translation control sequences such that the template nucleic acids are capable of being translated, or transcribed and translated using translation and/or transcription effectors. Transcription and translation control sequences can be of any species so long as they allow for transcription from DNA to mRNA and for translation from mRNA to protein, and can be suitably selected according to the species of the transcription and translation effectors. The transcription control and translation control sequences may exist as separate regions or may overlap on the template nucleic acid.

Transcription control sequences can include, for example, one or more of promoter, terminator, and enhancer sequences. For example, a free template nucleic acid can include promoter and terminator sequences. The promoter sequence used in the template nucleic acid is dependent upon the choice of transcription effector. “Transcription effector” refers to a composition capable of synthesizing RNA from an RNA or DNA template, e.g., a RNA polymerase, and includes nucleotide triphosphates (NTPs). For example, a transcription effector can be a prokaryotic phage RNA polymerase such as a T7, T3, or SP6 RNA polymerase. As such, if a T7 RNA polymerase is to be used as a transcription effector, the template nucleic acid sequence contains a promoter sequence recognized by the T7 RNA polymerase.

Translation control sequences can include ribosome binding sites such as the Kozak sequence (A/GCCACCAUGG, SEQ ID NO:1) or the Shine-Dalgarno (SD) sequence (AGGAGG). In embodiments in which eukaryotic translation effectors are used, a template nucleic acid can lack a Kozak sequence if the 5′-untranslated region (UTR) lacks stable secondary structure. The term “translation effector” refers to a macromolecule capable of decoding a messenger RNA and forming peptide bonds between amino acids. The term encompasses ribosomes, and catalytic RNAs with the aforementioned property. A translation effector can optionally further include tRNAs, tRNA synthases, elongation factors, initiation factors, and termination factors. In one embodiment, the translation effector is a prokaryotic or eukaryotic cell lysate or extract. For example, a prokaryotic cell lysate or extract can be an Escherichia coli extract. A eukaryotic cell lysate or extract can be rabbit reticulocyte lysate or wheat germ extract.

A template nucleic acid further can include one or more of an untranslated leader sequence, a sequence encoding a cleavage site, a recombination site, a 3′ untranslated sequence, or an internal ribosome entry site.

### Particles

Polypeptides are produced using the free template nucleic acid and transcription and/or translation effectors in the presence of particles such that the polypeptide can be captured during its synthesis. For example, when the template nucleic acid is DNA, transcription and translation effectors are included with the particles to produce the polypeptide. When the template nucleic acid is mRNA, translation effectors are included with the particles to produce the polypeptide. Suitable particles range in size from 0.8 to 3.0 μm in diameter. In some embodiments, the particles are magnetic. Alternatively, non-magnetic, filterable particles can be used such as those in the diameter range of 40-100 micron. For example, MyOne™ Dynald® beads can be used. In some embodiments, the polypeptide can be captured on a particle via the hydrophobic surface of the particle without the need for an agent having binding affinity for the tag. In some embodiments, hydrophilic particles are coated with an agent having binding affinity for the tag on the encoded polypeptide such that the polypeptide can be captured on the particle. The particles containing the bound polypeptides can be separated from transcription and translation effectors. For example, when the particles are magnetic, a magnet can be used to separate the particles from the other components in the reaction. The particles containing the bound polypeptides then can be used, e.g., in a biological assay or to form arrays as described herein.

In some embodiments, the amount of polypeptide produced using the transcription and translation effectors can be determined. For example, if the tag is a fluorescent protein, the amount of fluorescence can be measured to quantitate the amount of polypeptide produced. For the purpose of detecting the presence of the polypeptide, a mutant fluorescent tag can be used such as superfolder GFP or reporter GFP. See Pédelacq et al., Nat. Biotechnol., 24(1):79-88 (2006); and Waldo et al., Nat. Biotechnol. 17(7):691-5 (1999). The amount of fluorescence can be measured using, for example, a microarray reader, microscope, or microfluidic device capable of detecting fluorescence.

In some embodiments, the methods described herein use a particle coated with an agent such that the particle has binding affinity for one tag (e.g., a fluorescent tag). In some embodiments, the methods described herein use a plurality of different free template nucleic acids encoding polypeptides with different tags and a plurality of different particles, wherein each different particle has binding affinity for one tag. In some embodiments, the methods described herein use a particle coated with two or more different agents such that the particle has binding affinity for two or more tags (e.g., fluorescent tags).

The agent coated on a particle can be, for example, an antibody or antigen binding fragment thereof, an aptamer, or synthetic antibody (“synbody” see below). “Antibody” as the term is used herein refers to a protein that generally includes heavy chain polypeptides and light chain polypeptides. IgG, IgD, and IgE antibodies comprise two heavy chain polypeptides and two light chain polypeptides. IgA antibodies comprise two or four of each chain and IgM antibodies generally comprise 10 of each chain. Single domain antibodies having one heavy chain and one light chain and heavy chain antibodies devoid of light chains are also contemplated. A given antibody comprises one of five types of heavy chains, called alpha, delta, epsilon, gamma and mu, the categorization of which is based on the amino acid sequence of the heavy chain constant region. These different types of heavy chains give rise to five classes of antibodies, IgA (including IgA1 and IgA2), IgD, IgE, IgG (IgG1, IgG2, IgG3 and IgG4) and IgM, respectively. A given antibody also comprises one of two types of light chains, called kappa or lambda, the categorization of which is based on the amino acid sequence of the light chain constant domains.

“Antigen binding fragment” of an antibody refers to an antigen binding molecule that is not a complete antibody as defined above, but that still retains at least one antigen binding site. Antibody fragments often include a cleaved portion of a whole antibody, although the term is not limited to such cleaved fragments. Antigen binding fragments can include, for example, a Fab, F(ab)₂, Fv, and single chain Fv (scFv) fragment. An scFv fragment is a single polypeptide chain that includes both the heavy and light chain variable regions of the antibody from which the scFv is derived. Other suitable antibodies or antigen binding fragments include linear antibodies, multispecific antibody fragments such as bispecific, trispecific, and multispecific antibodies (e.g., diabodies (Poljak, Structure 2(12):1121-1123 (1994); Hudson et al., J. Immunol. Methods 23(1-2):177-189 (1994)), triabodies, tetrabodies), minibodies, chelating recombinant antibodies, intrabodies (Huston et al., Hum. Antibodies 10(3-4):127-142 (2001); Wheeler et al., Mol. Ther. 8(3):355-366 (2003); Stocks, Drug Discov. Today 9(22): 960-966 (2004)), nanobodies, small modular immunopharmaceuticals (SMIP), binding-domain immunoglobulin fusion proteins, camelid antibodies, camelized antibodies, and V_{HH} containing antibodies.

The term “aptamer” refers to small peptides or oligonucleotides that specifically bind to a target molecule. Such aptamers can be identified using various selection protocols. For example, an oligonucleotide aptamer can be identified, for example, using “Systematic Evolution of Ligands with EXponential enrichment” (SELEX) or microfluidic SELEX, and a library of synthetically derived random nucleic acid molecules (e.g., 30 to 60, 35 to 45, or 40 nucleotides in length). SELEX uses alternate cycles of ligand selection from pools of variant sequences and amplification of the bound species. Multiple rounds exponentially enrich the population for the highest affinity species that can be clonally isolated and characterized. See, for example, Ellington and Szostak, Nature, 346:818-822 (1990); Tuerk and Gold, Science, 249(4968):505-510 (1990); Stoltenburg et al., Biomol Eng., 24(4):381-403 (2007); and Cho et al., Proc. Natl. Acad. Sci. USA, 107(35): 15373-15378 (2010).

Peptide aptamers can be selected, for example, by expressing a combinatorial library of constrained peptides that are 10 to 35 amino acids (e.g., 15 to 25 or 20 amino acids) in length such that the peptides are displayed from a surface loop of a scaffold protein (e.g., thioredoxin, GFP, Staphylococcus nuclease, or SteA). High-throughput systems such as the yeast two-hybrid or retroviral delivery to mammalian cells can be used to identify individual peptides that specifically bind the target. See, Miller et al., J. Mol. Biol., 365(4):945-957 (2007).

Peptide aptamers also can be selected using a peptide array containing randomly generated peptides (e.g., 10,000 randomly generated peptides) that are 10 to 35 amino acids (e.g., 15 to 25 or 20 amino acids) in length. The peptide array can be screened using a binding assay with the protein target (e.g., GFP or thioredoxin). For example, when a fluorescent protein such as GFP is the target, GFP-binding peptides can be directly identified through a fluorescent scanner after a GFP binding assay. Spots that show strong fluorescence contain peptides that bind specifically to the GFP. Using such an assay, the following peptides were identified: CSGFRAMWLYRNWESQVEAT (SEQ ID NO:2), CSGWNHVIYEGTRYNWFRDS (SEQ ID NO:3), and CSGPYGTHFMYKSGGWRAIY (SEQ ID NO:4).

When thioredoxin is the target, an anti-thioredoxin IgG (e.g., a goat, mouse, or human anti-thioredoxin IgG) can be applied to the peptide array after the initial target binding step. This is followed by applying a secondary antibody (corresponding to the primary anti-thioredoxin IgG), tagged with a reporter molecule (e.g., a fluorescent reporter such as alexa fluor 647, alexa fluor 555, Cy-5, Cy-3, or other fluorescent tag).

Thioredoxin-binding peptides can be identified through a fluorescence scanner. Spots that show strong fluorescence contain peptides that bind specifically to thioredoxin. Using such an assay, at least six peptides were identified. See Table 1.

TABLE 1
Thioredoxin-binding peptides

| | | | | Ext. | SEQ |
|---|---|---|---|---|---|
| Name | Sequence | MW | PI | Coef | ID |
| TRX1 | LVTDETISYFRDQDAEIGSC | 2262.8 | 3.4 | 1490 | 5 |
| TRX2 | IIHWKQYHADMLLLEWKGSC | 2471.9 | 7.3 | 12490 | 6 |
| TRX3 | TPPLSSRWEHWFNMQNKGSC | 2405.7 | 9.0 | 11000 | 7 |
| TRX4 | WWYTLGEQIPRWPQKGWGSC | 2478.8 | 8.9 | 23490 | 8 |
| TRX5 | IQEWSNMVIWQETYRKIGSC | 2471.8 | 6.4 | 12490 | 9 |
| TRX6 | PGKDRADWKHYGNYYPTGSC | 2315.5 | 8.7 | 9970 | 10 |

Peptide aptamers identified using any of the methods described herein can be subjected to mutagenesis to improve the affinity and specificity of the peptide.

Synbodies also can be used to capture the tagged polypeptides. Using a synbody instead of an antibody can eliminate cross reactions between anti-immunoglobulins and reduce the cost of arrays produced using the methods described herein. Synbodies can be made by linking two or more target binding peptides (e.g., identified as described above) to one another to form a multimer. See, for example, WO 2009140039, WO 2010111299, and Diehnelt et al., PLoS One. 5(5):e10728 (2010). A pair of peptides can be joined to one another with one linker in four orientations (N-terminus to N-terminus, C-terminus to C-terminus, N-terminus to C-terminus and C-terminus to N-terminus). The orientation of linkage can be controlled by the reactive groups at the termini of the peptides and the linker. One, some, or all of the possible orientations can be synthesized. In some methods, a pair of peptides is joined to one another by two linkers forming a cyclic structure. Again multiple orientations of the same peptides can be joined in a cyclic structure. For example, two peptides can be joined N-terminus to N-terminus and C-terminus to C-terminus, or N-terminus to C-terminus and C-terminus to N-terminus or vice versa.

Suitable linkers can be peptidic or nonpeptidic (e.g., DNA or PEG). The linker can also be an amino acid flanked by PEG on both sides. Optionally, a library of linkers can be synthesized on beads by a split-pool approach (see, e.g., Burbaum et al., Proc Natl Acad Sci USA. 92(13):6027-31 (1995)). The linkers typically vary in length, flexibility, charge, or charge distribution. The length can be controlled by the number of amino acids or other monomers in a polymeric linker. The length can vary from about 0.1 nm (in the case of direct bonding of one peptide to another by a non-peptidic bond) to about 30 nm. The flexibility can be controlled by the number of proline residues (the more proline residues, the more rigid the linker). Proline and glycines are relative inert with respect to potential interactions with a target. The charge can be controlled by the number and distribution of charged residues. Positively charged residues include arginine, lysine and sometimes histidine. Negatively charged amino acids include glutamate and aspartate. The linkers can also have a branched structure (e.g., multi-antigenic MAP linkers) to form multimers with more than two peptides. A simple example of a MAP linker is a lysine residue in which peptides are attached to alpha and epsilon moieties of the lysine.

One example of a linker is a polyproline or poly (proline glycine proline) in which one or both distal portions of the linker are azido-modified to facilitate conjugation to one or more peptides by azide-alkyne conjugation. Alternatively, such linkers can be alkyne-modified on one or both terminal residues and conjugated to azido-modified peptides. Another example of a linker has the formula (pro pro X pro pro) n, wherein X is an amino acid that varies between linkers and n is between 1 and 10. Other linkers have propargyl lysine residues as the C- or N-terminal residue or residue adjacent to the C- or N-terminal residue.

The linker plays a role of holding the two peptides together in such a manner that both peptides can interact with their respective binding sites on a target. The length of linker depends on the relative spacing of binding sites on the target. Typically, a minimum length of linker is needed for both binding peptides to bind simultaneously. Thus, if the length of linker is increased for a given peptides, the binding typically shows a steep increase as the minimum length of linker is reached, plateaus and then gradually decreases as the linker length is increased. A more flexible linker typically increases the on-rate and off-rate of a multimer. Because a high on-rate and a low-off rate are usually desired, there is usually an optimum flexibility of a linker for a particular peptide pair. As well as holding two peptides together, a linker can also contribute to binding to the target, particularly via the inclusion of charged amino acids in the linker.

### Methods of Producing Arrays

Particles containing the bound polypeptides can be used to make arrays (e.g., high density arrays) on solid substrates such as glass slides or microchips with an amine-reactive surface. Such glass slides are commercially available, for example, from Surmodics, Inc. (Codelink slides) and Schott. Aminosilane slides functionalized with aldehyde functions also can be used for immobilization of polypeptide-carrying particles. Polypeptide bound particles described herein can be dried and rewet without loss of fluorescence or folding, which is particularly useful for automatic printing and imaging (e.g., using the Typhoon™ Imaging system from Amersham Pharmacia Biotech).

The particles containing the bound polypeptides can be washed in a buffer (e.g., phosphate buffered saline, pH 7.4) and then can be washed and resuspended in spotting buffer having a pH of 8.5 or higher. The spotting buffer can be 0.1 M phosphate or sodium carbonate, with 0 to 30% glycerol (e.g., 5% glycerol) or polyvinyl alcohol (PVA). The glycerol or PVA is used to adjust the viscosity of the solution for maintaining the particle suspension such that during spotting, covalent crosslinking between the particles and array surface can occur before the spotting buffer dries.

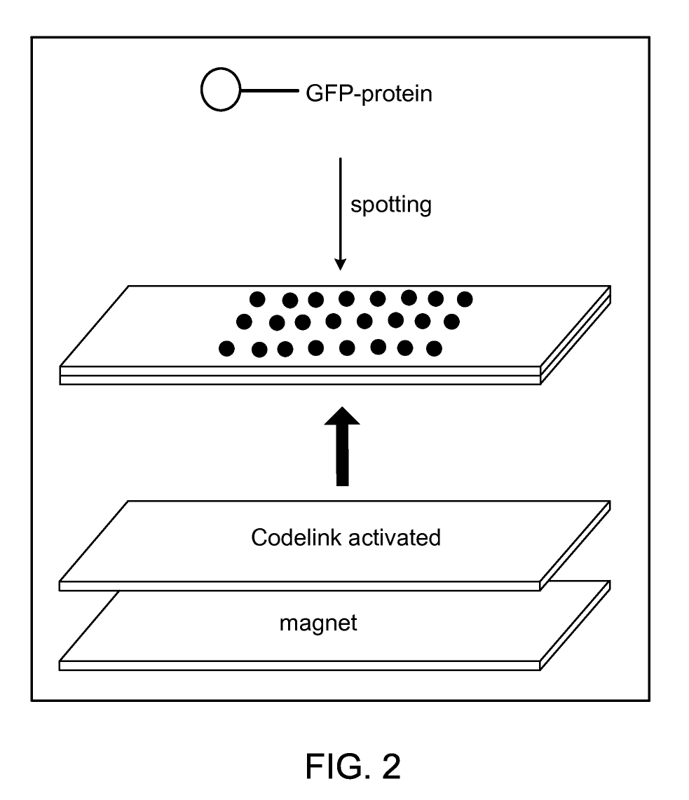

A spotter (e.g., from Perkin Elmer), nanoprint spotter (e.g., from Arryit Corporation), or a piezo spotter (e.g., from Aurigintech) can be used to spot the particles onto the solid substrate. Humidity in the spotting chamber must be maintained at higher than 50%. After spotting, the solid substrate (e.g., glass slide) is kept in the humidity chamber (humidity>50%) over night for immobilization reactions. To facilitate the immobilization process, the slides can be placed on a magnet that is approximately the same size as the slide. See, FIG. 2 for a schematic of the immobilization of the protein-bound particles onto a slide using a magnet based slide holder. Using such a magnet based slide holder can prevent the particles from dispersing to other areas on the slide surface.

In some embodiments, an acoustic delivery system (e.g., from Nextval, San Diego, Calif.) is used to print the high-density particles. Such a system uses constant agitation, which eliminates settling that can occur with standard contact or piezo spotting techniques.

After immobilizing the protein-bound particles on the substrate, by any of the above methods, the surfaces can be washed with buffer (e.g., TBST buffer containing 50 mM Tris HCl, pH 7.4, 150 mM NaCl, and 0.1% Tween 20) to remove non-immobilized particles. In some embodiments (e.g., when an antibody is used as the capture agent), the surface can be blocked with a blocking buffer containing, e.g., 3% bovine serum albumin (BSA) or milk before assaying. In embodiments in which the tag is a fluorescent protein, the amount of protein at each spot can be estimated by scanning the slide for fluorescence using a fluorescence scanner.

In embodiments in which the tag is thioredoxin, the amount of protein at each spot can be estimated by applying a solution of anti-thioredoxin IgG (e.g., goat, mouse, or human anti-thioredoxin IgG) to the array surface, followed by a solution containing a fluorescently labeled secondary antibody corresponding to the primary anti-thioredoxin IgG. A fluorescence scanner then can be used to scan the slide for fluorescence.

The invention will be further described in the following examples, which do not limit the scope of the invention described in the claims.

### EXAMPLES

### Example 1

### Capture of In Vitro Translated Fusion Proteins and Detection of Fluorescence without a Capture Agent

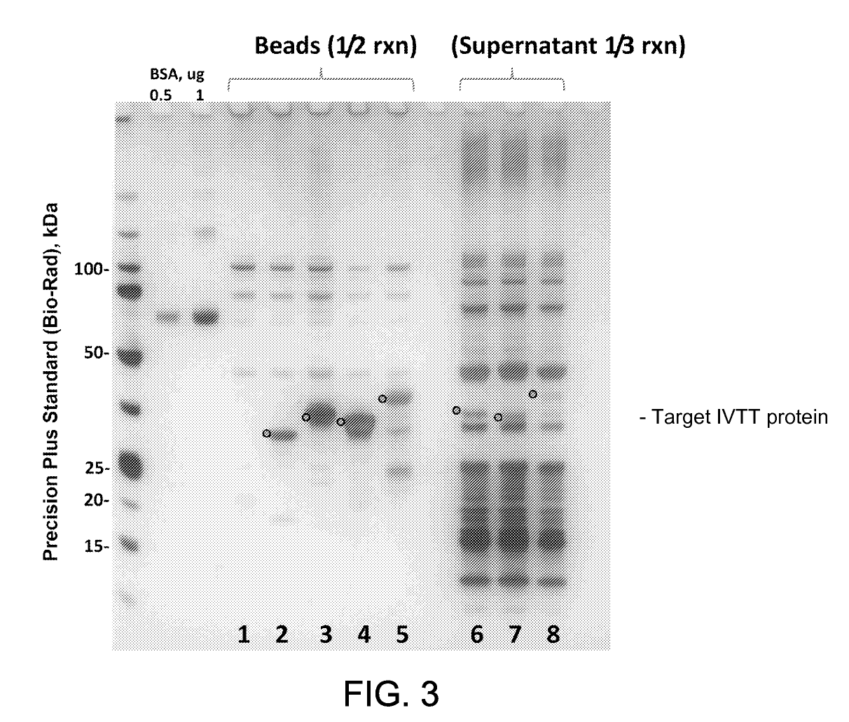

Two Franciscella tularensis (FTT) GFP-fusion proteins and two ASFV (African Swine Fever Virus) GFP-fusion proteins were produced by in vitro transcription and translation (IVTT) in the Expressway™ Mini Cell Free system (Invitrogen, Carlsbad, Calif.) using free template nucleic acid and tosylactivated M-280 magnetic beads (Invitrogen). The capture-agent-free beads were added during the synthesis reaction; this is in contrast to the standard procedure of adding beads, with capture agents attached, subsequent to synthesis for polypeptide capture. The translation products were washed and subjected to SDS-PAGE, and the resulting gel was Coomassie stained. See FIG. 3. In the SDS-PAGE Coomassie stained gel, lanes 1 and 2 show concentration standards (BSA). Lanes 3-5 are the washed beads following IVTT reactions. Lanes 6-8 are the supernatant of the IVTT reaction before washing. These wells display the unbound proteins of the reaction mix. The dots mark the position corresponding to the calculated molecular weight of the fusion protein. Note there is no polypeptide band corresponding to the target protein molecular weight in the supernatant lanes, indicating quantitative capture by the beads.

Next, 20 different FTT genes predicted or known to encode membrane proteins were selected for synthesis in both the standard and the new bead-based systems. This experiment was conducted using ³⁵S-labeled FTT-thioredoxin/6× his tagged fusion proteins produced by i) the new method through IVTT with free template nucleic acid and translation products captured during synthesis onto hydrophobic magnetic particles (Dynal MyOne) without any capture agent attached or ii) the currently practiced protocol: using well described methods of synthesizing polypeptides and then capturing the polypeptides onto magnetic particles (subsequent to synthesis) containing an anti-tag capture agent for purification, as described above. In this experiment, in vitro expression of F. tularensis proteins were performed using PURExpress™ In vitro Protein Synthesis Kit (New England, BioLabs®, Inc) in 96-well format. Approximately 250 ng of DNA template were used for a 254, IVTT reaction. The transcription and translation procedures were carried out following the manufacture protocols. To enable autoradiographs, 10 μCi ³⁵S labeled methionine was added into each IVTT reaction. The reactions were incubated in Gene Machines HiGro Orbital Incubator for one hour with shaking at 650 rpm. After the reactions were complete, supernatants were removed using 96-well magnetic separator (MagnaBot® 96 Magnetic Separation Device, Promega).

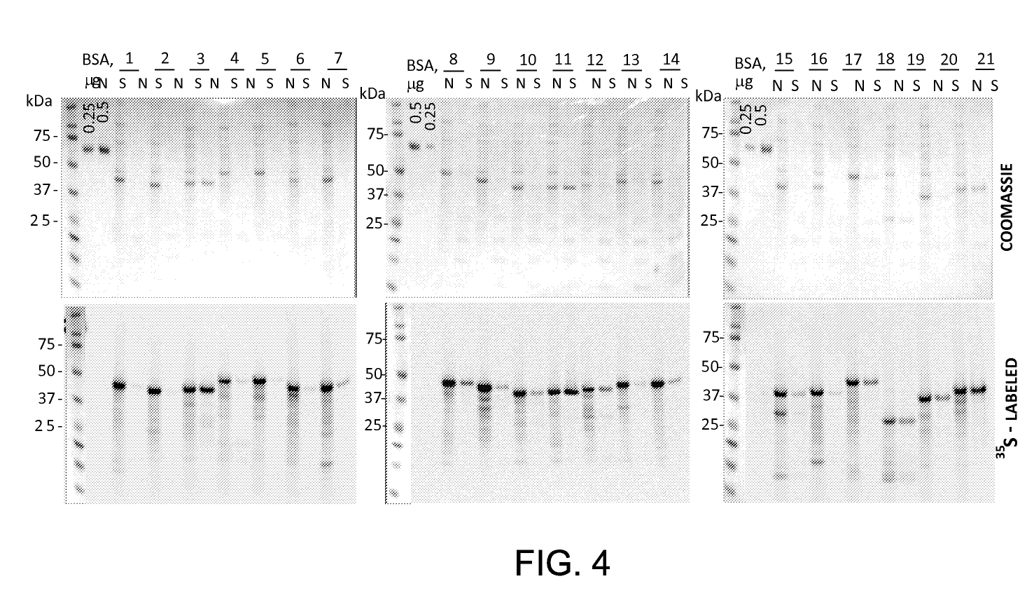

In this experiment, nickel coated beads were used as capture agent for the His-tagged IVTT products. However, bead-conjugated anti-tag antibodies also can be used. The translation products were washed and subjected to SDS-PAGE, and the resulting gel was Coomassie stained. After visualization of the stain, the gel was dried and prepared for phosphorimaging of the radioactivity. Only 10% of the products from i) were loaded onto the gel while 20% of the products from ii) were loaded onto the gel to facilitate visualization of the lower yielding reactions. The yield of translation products can be estimated by comparison to the BSA concentration standards fractionated between the molecular weight standards and the IVTT products (lanes 2 and 3 of each gel). The results in FIG. 4 show that yields are consistently higher using the new co-translational, capture-agent free protocol. In fact a number of the polypeptides, loaded at twice the sample volume relative to the new method, are not even measurably produced by the standard method.

It was found that the nascent polypeptide chains bind highly selectively to the magnetic beads, even without additional capture agent. Since the polypeptide cannot be eluted with low pH glycine, but can be eluted with mild detergents, it is thought that the hydrophobic surfaces of the extended polypeptide chain interact with the hydrophobic surface of the beads. This indicates that capture does not require monoclonal capture antibodies. As a result, the target protein bound bead samples are more pure, i.e., the samples do not include immunoglobulins. Capture via the hydrophobic surface of the bead was more efficient than capture with an antibody (e.g., anti-thioredoxin or anti-HIS antibody).

### Example 2

### Assessment of Different Linkers

Four FTT GFP-fusion proteins and 3 ASFV GFP-fusion proteins (row 3, spots 1-3) were produced by IVTT using the Expressway™ Mini Cell Free system (Invitrogen, Carlsbad, Calif.) and bound to tosylactivated M-280 magnetic beads (Invitrogen). No capture agent was used on the particles. The IVTT reactions (2 μl) were spotted onto aminosilane-coated glass slides, and allowed to dry. The slide was then scanned in a Typhoon™ scanner for GFP fluorescence indicative of the presence of natively folded protein. For the FTT target genes, four separate versions of the GFP fusion protein were made per target gene. For the ASFV GFP fusion proteins, only the linker GSAGSAAGSGEF (SEQ ID NO:12; see Waldo, et al., Nat. Biotechnol. 1999 17(7):691-5) was tested (row 3).

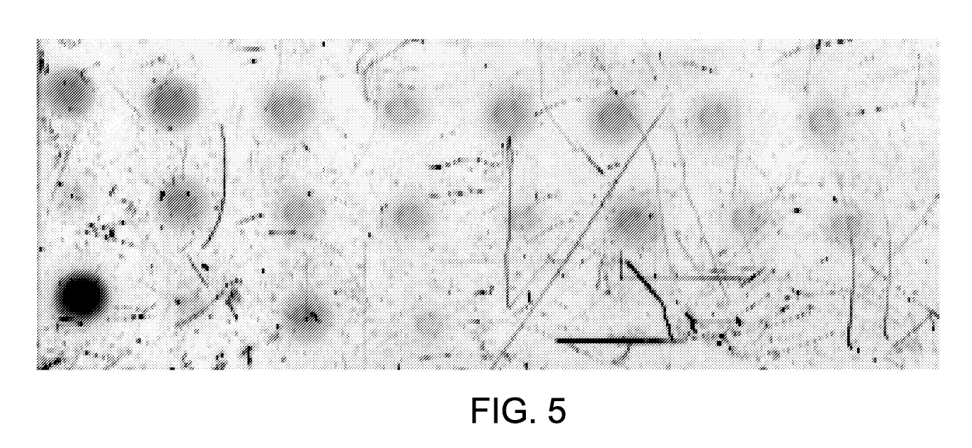

FIG. 5 contains the results from the scanner. In FIG. 5, row 1 shows the GFP fluorescence from the fusion proteins carrying the linker TQPPSHGSAGSAAGSGEF (SEQ ID NO:11) between the FTT protein and GFP both with (row 1, spots 1-4) and without hemagglutinin (HA) and His tags (row 1, spots 5-8). Shown next in FIG. 5 are the same proteins fused using the linker having the amino acid sequence of SEQ ID NO:12 between the FTT protein and GFP both with (row 2, spots 1-4) and without HA and His tags (row 2, spots 5-8). There was no significant difference in observed fluorescence between the linkers. This demonstrates that there is flexibility in linker sequence selection.

The spotted IVT reactions from FIG. 5 also were examined by fluorescence confocal microscopy. A negative control IVT reaction that contained no template did not produce any GFP signal. For both FTT membrane protein and ASFV membrane protein GFP fusions, GFP signal was observed, indicating natively folded protein within the spot.

### Example 3

### Effect of Freezing on GFP Signal Observed by Fluorescence Microscopy

An ASFV membrane protein fused to GFP was produced by IVTT in the Expressway™ Mini Cell Free system (Invitrogen, Carlsbad, Calif.) and bound to tosylactivated M-280 magnetic beads (Invitrogen). No capture agent was used. Immediately upon completion of the IVTT reaction, 2 μl of the reaction were spotted on an aminosilane-coated glass slide and allowed to dry. The remaining IVTT reaction was frozen at −20° C., thawed, and 2 μl was spotted on the aminosilane-coated glass slide and allowed to dry. The spots were then examined for GFP fluorescence by confocal microscopy. It was determined that the fresh IVTT reaction had significantly higher GFP fluorescence than the frozen and thawed IVTT reaction.

### Example 4

### GFP Fusion Proteins Bound to Magnetic Beads can be Dried and Rewet without Loss of Fluorescence (and Thus Folding)

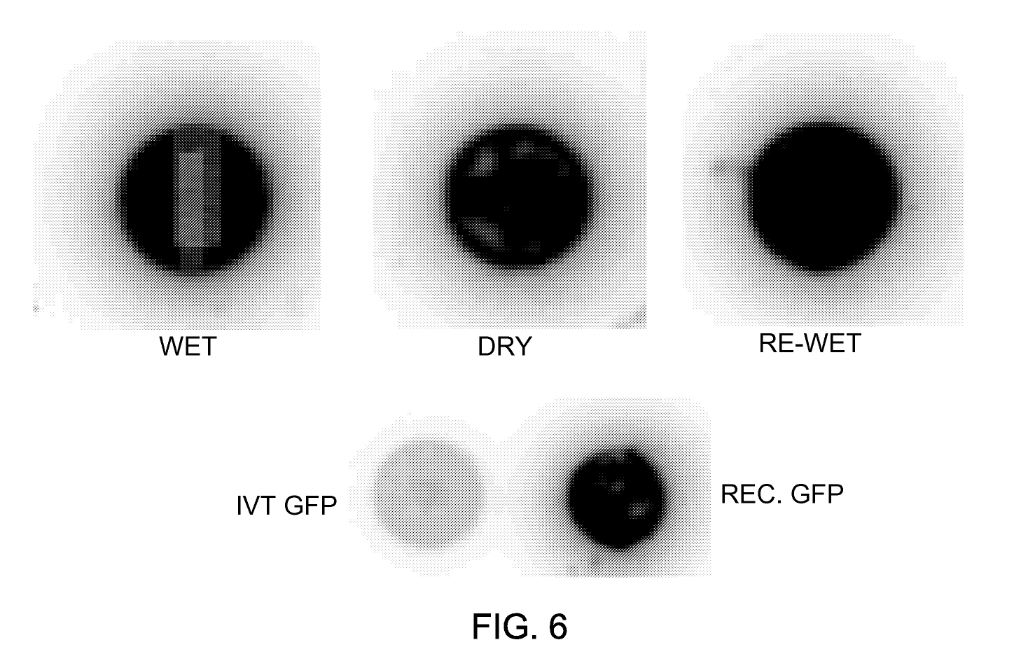

An ASFV membrane protein fused to GFP was produced by IVTT using the Expressway™ Mini Cell Free system (Invitrogen, Carlsbad, Calif.) and bound to tosylactivated M-280 magnetic beads (Invitogen). No capture agent was used. Immediately upon completion of the IVTT reaction, 2 μl of the reaction were spotted on an aminosilane-coated glass slide, and fluorescence levels were determined by a Typhoon™ scanner while the spot was still wet (FIG. 6, left panel), after it had been allowed to dry (FIG. 6, middle panel), and after it had been rewet by addition of 2 μl of 1X phosphate buffered saline (FIG. 6, right panel). The fluorescence levels were the same for all samples, indicating that the GFP-fusion protein remains properly folded through the drying and rewetting steps used in the automated printing of bead-bound proteins onto slides.

### Example 5

### Fusion Protein Capture onto Hydrophilic Magnetic Beads with an Anti-Tag Capture Agent

In this experiment, it is demonstrated that a protocol employing an anti-tag agent bound to beads for IVTT product capture is improved by the modification shown here of co-translational purification. An anti-thioredoxin antibody conjugated to magnetic beads was used for polypeptide capture as described below. However, it will be appreciated that other capture agents can be similarly employed.

i. Prepare of Anti-Thioredoxin (Anti-Trx) Bound Magnetic Beads

Magnetic Tosylactivated beads were purchased from Invitrogen (Dynabeads® M280 Tosylactivated). Beads were equilibrated by washing three times with 500 μl of buffer containing 2.4M (NH₄)₂SO₄ and 1.0M H₃BO₃. Equilibrating buffer was removed using a magnetic bead separator (Invitrogen). Anti-thioredoxin antibody (1 μg/μL) was coupled to beads with a ratio of 1:1.67 antibodies to volume of beads accordingly. An equal antibody volume of buffer was added to the coupling reaction to a final concentration of 1.2M (NH₄)₂SO₄ and 0.5M H₃BO₃. The reaction was incubated at 37° C. overnight with shaking at 990 rpm (Roche, Proteomaster Rapid Translation System). After incubation, supernatant was removed and beads were blocked with 0.5% BSA in PBS for 1 hour with shaking at 37° C. Before any use, antibody-coupling beads were washed 3 times with PBS and stored at 4° C. For in vitro translation of 84 samples per 96-well plate, 2.1 mL of tosylactivated beads and 1.26 mL of anti-thioredoxin antibody was used for each time.

ii. In Vitro Transcription, Translation, and Purification in 96-Well

In vitro expression of F. tularensis proteins was performed using PURExpress™ In vitro Protein Synthesis Kit (New England, BioLabs®, Inc) in 96-well format. Approximate 250 ng of DNA template was used for a 254, IVTT reaction. The transcription and translation procedures were carried out following the manufacture protocols with an exception that IVTT reactions were run in the presence of anti-thioredoxin antibody coupled 1.0 μm or 2.8 μm magnetic beads. For autoradiographs, 10 μCi of ³⁵S labeled methionine were added into each IVTT reactions. The reactions were incubated in Gene Machines HiGro Orbital Incubator for one hour with shaking at 650 rpm. After the reactions were complete, supernatants were removed using a 96-well magnetic separator (MagnaBot® 96 Magnetic Separation Device, Promega). Protein-bound beads were then washed 3 times with PBS buffer. Beads were stored in PBS at −20° C. for further analysis.

iii. SDS PAGE Analyses

For SDS PAGE and autoradiograph analysis, Bio-Rad Criterion XT 26-well 4%-12% Bis-Tris precast gradient gels were used. IVTT proteins in 12.5 μA reaction bound on magnetic beads were eluted with 20 μA SDS containing 5% β-mercaptoethanol. The beads and denaturant were boiled for 5 minutes; then, beads were separated from the mixture by a magnetic separator (Invitrogen Dynal bead separations). Invitrogen SimplyBlue stain was used to visualize the bands from the gel. Approximate 5 μL of supernatant was spotted on glass fiber filter for TCA precipitation and yield determination. For imaging, acrylamide gel was dried under vacuum and transferred to phosphor screen. Autoradiograph of IVTT made proteins were visualized by Typhoon™ imaging (Molecular Dynamics).

### Example 6

### Printing the Bead-Bound IVTT Products onto Microarrays Acoustically

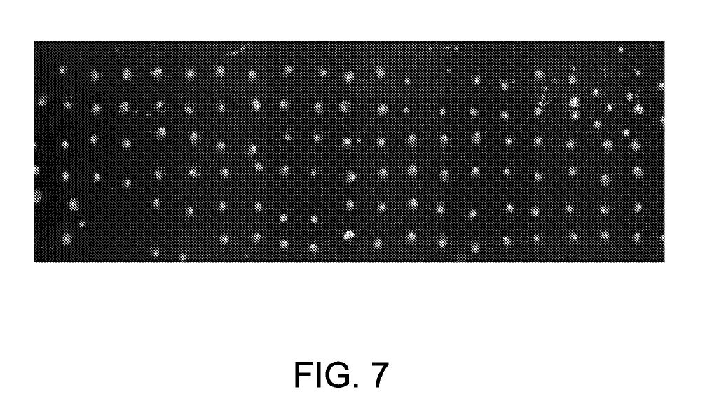

To improve the consistency of protein spot densities, the agitation that is part of an acoustic delivery process was used (Nextval, San Diego, Calif.). Bead-IVTT samples were continuously agitated during the streamline printing process. Sample sizes 2 nl or less were “shot” upward into an inverted functionalized slide, such as those described for contact and piezo printing. This afforded highly consistent printing quantities and uniform spot morphologies. See FIG. 7.

Quality and quantity of spotting of microarrays prepared using any of the methods described herein can be directly visualized when the polypeptide target is a fusion protein with GFP, a variant of GFP, or other fluorescent protein. In particular, some GFP fusions allow visualization of not all polypeptides, but only those that are properly folded (folding reporter GFP). Other GFP variants can be used to detect all samples regardless of the targets folded state, such as superfolder GFP, which will fluoresce even if fused to an unfolded protein. The array is then treated as described for analyte analyses.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for producing a polypeptide in vitro, said method comprising:
a) producing said polypeptide using a free template nucleic acid, a transcription effector, and a translation effector in the presence of a particle; said free template nucleic acid encoding said polypeptide and capable of being transcribed and translated; wherein said polypeptide comprises a tag, and wherein said particle optionally comprises a peptide or synbody having affinity for said tag; and
b) capturing said polypeptide on said particle during synthesis.

2. The method of claim 1, wherein said polypeptide is a membrane protein.

3. The method of claim 1, wherein said polypeptide is a hydrophobic polypeptide.

4. The method of claim 1, wherein said tag is a fluorescent tag.

5. The method of claim 4, wherein said fluorescent tag is green fluorescent protein (GFP) or enhanced GFP.

6. The method of claim 4, wherein said fluorescent tag is blue fluorescent protein, cyan fluorescent protein, red fluorescent protein, or yellow fluorescent protein.

7. The method of claim 1, wherein said particle is a magnetic particle.

8. The method of claim 1, wherein said tag is thioredoxin.

9. The method of claim 1, wherein said particle is a hydrophobic particle.

10. The method of claim 4, wherein a plurality of different template nucleic acids and a plurality of different particles are provided, wherein each said different template nucleic acid encodes a polypeptide having a different fluorescent tag; and wherein each said different particle has binding affinity for one fluorescent tag.

11. The method of claim 4, wherein a plurality of different template nucleic acids is provided; wherein each said different template nucleic acid encodes a polypeptide having a different fluorescent tag; and wherein each said particle has binding affinity for two or more fluorescent tags.

12. The method of claim 1, wherein a plurality of different template nucleic acids is provided; wherein each said different template nucleic acid encodes a polypeptide having a different tag.

13. The method of claim 1, further comprising separating said particle comprising said bound polypeptide from said transcription and translation effectors.

14. The method of claim 1, wherein said transcription effector is a prokaryotic RNA polymerase.

15. The method of claim 14, wherein said prokaryotic RNA polymerase is a T7, T3, or SP6 RNA polymerase.

16. The method of claim 1, wherein said translation effector is a prokaryotic or eukaryotic cell lysate or extract.

17. The method of claim 16, wherein said prokaryotic cell lysate or extract is an Escherichia coli extract.

18. The method of claim 16, wherein said eukaryotic cell lysate or extract is human, rabbit reticulocyte lysate, or wheat germ extract.

19. The method of claim 4, further comprising detecting fluorescence of said polypeptide bound to said particles.

20. The method of claim 19, further comprising measuring the amount of fluorescence to quantitate the amount of polypeptide produced using said transcription and translation effectors.

21. The method of claim 20, wherein the amount of fluorescence is measured using a microarray reader or microscope capable of detecting fluorescence.

22. The method of claim 20, wherein the amount of fluorescence is detected by a microfluidic device.

23. The method claim 1, further comprising spotting said particles comprising said bound polypeptide onto an amine reactive array surface or microchip.

24. The method of claim 4, wherein said fluorescent tag is at the C-terminus of said polypeptide.

25. The method of claim 1, wherein said particle comprises said peptide, and wherein said peptide is 10 to 30 amino acids in length.

26. A method for producing a polypeptide in vitro, said method comprising:
a) producing said polypeptide using a free template nucleic acid, a transcription effector, and a translation effector in the presence of a particle; said free template nucleic acid encoding said polypeptide and capable of being transcribed and translated; wherein said polypeptide comprises a fluorescent tag, and wherein said particle optionally comprises a peptide or synbody having binding affinity for said fluorescent tag;
b) capturing said polypeptide on said particle during synthesis; and
c) measuring the amount of fluorescence to quantitate the amount of polypeptide produced using said transcription and translation effectors.

27. The method of claim 26, wherein the amount of fluorescence is measured using a microarray reader or microscope capable of detecting fluorescence.

28. The method of claim 26, wherein the amount of fluorescence is detected by a microfluidic device.

29. A method for producing a polypeptide in vitro, said method comprising:
a) producing said polypeptide using a free template nucleic acid, a transcription effector, and a translation effector in the presence of a particle; said free template nucleic acid encoding said polypeptide and capable of being transcribed and translated; wherein said polypeptide comprises a tag; said particle comprising an agent having binding affinity for said tag; and
b) capturing said polypeptide on said particle via binding of said tag on said polypeptide to said agent on said particle.

30. The method of claim 29, wherein said agent is an antibody or antigen-binding fragment thereof.

31. The method of claim 30, wherein said antibody fragment is a Fab, F(ab′)2, Fv, or single chain Fv (scFv) fragment.

32. The method of claim 29, wherein said tag is thioredoxin.

33. The method of claim 29, wherein said tag is a fluorescent protein.
